(19) United States
(12) Patent Application Publication
(10) Pub. No.: US 2005/0042621 A1
(43) Pub. Date: Feb. 24, 2005

(54) Method for preparing a nucleic acid sample for hybridization to an array

(75) Inventor: Kyle B. Cole, Stanford, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/674,658

(22) Filed: Sep. 29, 2003

Related U.S. Application Data
(60) Provisional application No. 60/414,208, filed on Sep. 27, 2002.

(51) Int. Cl. C12Q 1/68; C12P 19/34

(52) U.S. Cl. 435/6; 435/91.2

(57) ABSTRACT

In one aspect of the present invention, a method for preparing a nucleic acid sample for hybridization to a nucleic acid array is presented. This disclosed method has the steps of providing a nucleic acid sample, the sample having mRNA, amplifying the mRNA to produce cRNA and fragmenting the cRNA with an RNase enzyme to produce fragments. In another aspect of the present invention, a method is provided for detecting hybridization of a nucleic acid sample to a nucleic acid array. In yet another aspect of the present invention, a method is presented for preparing cRNA for hybridization to an oligonucleotide probe array. In another aspect of the present invention, a method is provided for labeling an RNA sample, the method comprising providing an RNA sample, fragmenting the sample with an RNase enzyme to produce fragments and end-labeling the fragments with a detectable label. In an other aspect of the present invention, a method to detect an RNA molecule in an RNA sample is provided in which the end-labeled RNA fragments are hybridized a nucleic acid array.

### RELATED APPLICATIONS

This application claims priority to provisional application No. 60/414,208 filed Sep. 27, 2002, which is incorporated herein by reference in its entirety.

### BACKGROUND OF THE INVENTION

Many biological functions are accomplished by altering the transcriptional profile of various genes. For example, fundamental biological processes such as cell cycle progression, cell differentiation and cell death, are often characterized by variations in gene expression levels.

Nucleic acid hybridizations are commonly used in biochemical research and diagnostic assays. Generally a single stranded analyte nucleic acid is hybridized to labeled nucleic acid probe, and resulting nucleic acid duplexes are detected. Radioactive and nonradioactive labels have been used. Methods also have been developed to amplify the signal that is detected. Avidin-biotin systems have been developed for use in a variety of detection assays. Methods for the detection and labeling of nucleic acids in biotin systems are described, for example, in “Nonradioactive Labeling and Detection Systems”, C. Kessler, Ed., Springer-Verlag, New York, 1992, pp. 70-99; and in “Methods in Nonradioactive Detection,”, G. Howard, Ed., Appleton and Lange, Norwalk, Conn. 1993, pp. 11-27 and 137-150.

### SUMMARY OF THE INVENTION

In one aspect of the present invention, a method for preparing a nucleic acid sample for hybridization to a nucleic acid array is presented. This disclosed method has the steps of providing a nucleic acid sample, the sample having mRNA, amplifying the mRNA to produce cRNA and fragmenting the cRNA with an RNase enzyme to produce fragments. Preferably, this disclosed method has the additional step of hybridizing the fragments to an oligonucleotide probe array. In another embodiment, the RNase enzyme is preferably selected from the group consisting of Ribonuclease III, Nuclease S1, RNase A, RNase H, RNase T1, and Mung Bean nuclease. According to the present invention, fragments preferably have an average size of between about 20 to 500 nucleotides, more preferably between about 25 to 200 nucleotides and most preferably between about 50 to 100 nucleotides.

In another aspect of the present invention, a method is provided for detecting hybridization of a nucleic acid sample to a nucleic acid array. In yet another aspect of the present invention, a method is presented for preparing an RNA sample for hybridization to a nucleic acid array.

### BRIEF DESCRIPTION OF THE DRAWINGS

FIG. 1 is a photograph of a gel depicting the digestion of cRNA with Ribonuclease III and Nuclease S1.

FIG. 2 is a photograph of a gel depicting the gel shift of RNase III fragmented cRNA.

FIG. 3 shows digested cRNA for application to DNA microarrays.

FIG. 4 shows absolute calls of internally-labeled cRNA and end-labeled cRNA fragmented by magnesium hydrolysis and RNase III.

FIG. 5 shows scaled average signal of internally-labeled cRNA and end-labeled cRNA fragmented by magnesium hydrolysis and RNase III.

### DETAILED DESCRIPTION OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. patent application Ser. Nos. 10/063,559, 60/349,546, 60/376,003, 60/394,574, 60/403,381.

One of skill in the art will appreciate that in order to measure the transcription level (and thereby the expression level) of a gene or genes, it is desirable to provide a nucleic acid sample comprising mRNA transcript(s) of the gene or genes, or nucleic acids derived from the mRNA transcript(s). As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, suitable samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

In a particularly preferred embodiment, where it is desired to quantify the transcription level (and thereby expression) of a one or more genes in a sample, the nucleic acid sample is one in which the concentration of the mRNA transcript(s) of the gene or genes, or the concentration of the nucleic acids derived from the mRNA transcript(s), is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes. Where more precise quantification is required appropriate controls can be run to correct for variations introduced in sample preparation and hybridization as described herein. In addition, serial dilutions of “standard” target mRNAs can be used to prepare calibration curves according to methods well known to those of skill in the art. Of course, where simple detection of the presence or absence of a transcript is desired, no elaborate control or calibration is required.

In the simplest embodiment, such a nucleic acid sample is the total mRNA isolated from a biological sample. The term “biological sample”, as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes.

The nucleic acid (either genomic DNA or mRNA) may be isolated from the sample according to any of a number of methods well known to those of skill in the art. One of skill will appreciate that where alterations in the copy number of a gene are to be detected genomic DNA is preferably isolated. Conversely, where expression levels of a gene or genes are to be detected, preferably RNA (mRNA) is isolated.

Methods of isolating total mRNA are well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization with Nucleic Acid Probes, Part I. Theory and Nucleic Acid Preparation, P. Tijssen, ed. Elsevier, N.Y. (1993)).

In a preferred embodiment, the total nucleic acid is isolated from a given sample using, for example, an acid guanidinium-phenol-chloroform extraction method and polyA.sup.+ mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high density array may then include probes specific to the internal standard for quantification of the amplified nucleic acid.

One preferred internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of radioactivity (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al., Academic Press, Inc. N.Y., (1990).

Other suitable amplification methods include, but are not limited to polymerase chain reaction (PCR) (Innis, et al., PCR Protocols. A guide to Methods and Application. Academic Press, Inc. San Diego, (1990)), ligase chain reaction (LCR) (see Wu and Wallace, Genomics, 4: 560 (1989), Landegren, et al., Science, 241: 1077 (1988) and Barringer, et al., Gene, 89: 117 (1990), transcription amplification (Kwoh, et al., Proc. Natl. Acad. Sci. USA, 86: 1173 (1989)), and self-sustained sequence replication (Guatelli, et al., Proc. Nat. Acad. Sci. USA, 87: 1874 (1990)).

In a particularly preferred embodiment, the sample mRNA is reverse transcribed with a reverse transcriptase and a promoter consisting of oligo dT and a sequence encoding the phage T7 promoter to provide single stranded DNA template. The second DNA strand is polymerized using a DNA polymerase. After synthesis of double-stranded cDNA, T7 RNA polymerase is added and cRNA is transcribed from the cDNA template. Successive rounds of transcription from each single cDNA template results in amplified RNA. Methods of in vitro polymerization are well known to those of skill in the art (see, e.g., Sambrook, supra.) and this particular method is described in detail by Van Gelder, et al., Proc. Natl. Acad. Sci. USA, 87: 1663-1667 (1990) who demonstrate that in vitro amplification according to this method preserves the relative frequencies of the various RNA transcripts. Moreover, Eberwine et al. Proc. Natl. Acad. Sci. USA, 89: 3010-3014 provide a protocol that uses two rounds of amplification via in vitro transcription to achieve greater than 10⁶ fold amplification of the original starting material thereby permitting expression monitoring even where biological samples are limited.

It will be appreciated by one of skill in the art that the direct transcription method described above provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense as the target nucleic acids include both sense and antisense strands.

The protocols cited above include methods of generating pools of either sense or antisense nucleic acids. Indeed, one approach can be used to generate either sense or antisense nucleic acids as desired. For example, the cDNA can be directionally cloned into a vector (e.g., Stratagene's p Bluscript II KS (+) phagemid) such that it is flanked by the T3 and T7 promoters. In vitro transcription with the T3 polymerase will produce RNA of one sense (the sense depending on the orientation of the insert), while in vitro transcription with the T7 polymerase will produce RNA having the opposite sense. Other suitable cloning systems include phage lamda vectors designed for Cre-loxP plasmid subcloning (see e.g., Palazzolo et al., Gene, 88: 25-36 (1990)).

In a particularly preferred embodiment, a high activity RNA polymerase (e.g. about 2500 units/.mu.L for T7, available from Epicentre Technologies) is used.

Nucleic Acid Labeling

In a preferred embodiment, the hybridized nucleic acids are detected by detecting one or more labels attached to the sample nucleic acids. The labels may be incorporated by any of a number of means well known to those of skill in the art. However, in a preferred embodiment, the label is simultaneously incorporated during the amplification step in the preparation of the sample nucleic acids. For example, polymerase chain reaction (PCR) with labeled primers or labeled nucleotides will provide a labeled amplification product. The nucleic acid (e.g., DNA) is be amplified in the presence of labeled deoxynucleotide triphosphates (dNTPs). The amplified nucleic acid can be fragmented, exposed to an oligonucleotide array, and the extent of hybridization determined by the amount of label now associated with the array. In a preferred embodiment, transcription amplification, as described above, using a labeled nucleotide (e.g. fluorescein-labeled UTP and/or CTP) incorporates a label into the transcribed nucleic acids.

Alternatively, a label may be added directly to the original nucleic acid sample (e.g., mRNA, polyA mRNA, cDNA, etc.) or to the amplification product after the amplification is completed. Such labeling can result in the increased yield of amplification products and reduce the time required for the amplification reaction. Means of attaching labels to nucleic acids include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

In many applications it is useful to directly label nucleic acid samples without having to go through amplification, transcription or other nucleic acid conversion step. This is especially true for monitoring of mRNA levels where one would like to extract total cytoplasmic RNA or poly A+ RNA (mRNA) from cells and hybridize this material without any intermediate steps that could skew the original distribution of mRNA concentrations. See U.S. Pat. No. 6,344,316, which is hereby incorporated by reference in its entirety for all purposes.

In general, end-labeling methods permit the optimization of the size of the nucleic acid to be labeled. End-labeling methods also decrease any sequence bias associated with polymerase-facilitated labeling methods. End labeling can be performed using terminal transferase (TdT). End labeling can also be accomplished by ligating a labeled nucleotide or oligonucleotide or polynucleotide or analog thereof to the end of a target nucleic acid or probe. See U.S. Pat. No. 6,344,316.

This invention thus provides methods of labeling a nucleic acid and reagents useful therefore. Many of the methods disclosed herein involve end-labeling. Those skilled in the art will appreciate that the invention as disclosed is generally applicable in the chemical and molecular-biological arts.

Alkaline phosphatase may be used to remove the phosphate groups from the 3′ ends of the RNA fragments. The donor labeled ribonucleotide with a 5′-terminal phosphate is then ligated to the 3′ OH groups of the RNA fragments using T4 RNA ligase. T4 RNA ligase catalyzes ligation of a 5′ phosphoryl-terminated nucleic acid donor to a 3′ hydroxyl-terminated nucleic acid acceptor through the formation of a 3′ to 5′ phosphodiester bond, with hydrolysis of ATP to AMP and PPi. Although the minimal acceptor must be a trinucleoside diphosphate, dinucleoside pyrophosphates (NppN) and mononucleoside 3′,5′-disphosphates (pNp) are effective donors in the intermolecular reaction. See Hoffmann and McLaughlin, Nuc. Acid. Res. 15, 5289-5303 (1987), which is hereby incorporated by reference in its entirety for all purposes.

According to the present invention, a method is provided for preparing a nucleic acid sample for hybridization to a nucleic acid array, the method having the steps of providing a nucleic acid sample, the sample having mRNA; amplifying the mRNA to produce cRNA; and fragmenting the cRNA with an RNase enzyme to provide fragments. Preferably, according to the instant invention, a further step is carried out of hybridizing the fragments to an oligonucleotide probe array.

According to the present invention, an RNase enzyme is any enzyme which catalyzes the fragmentation or hydrolysis of RNA molecules or chains. Preferably, according to the present invention, the RNase enzyme is selected from the group consisting of Ribonuclease III, Nuclease S1, RNase A, RNase H, RNase T1, and Mung Bean nuclease. Most preferably, the RNase enzyme is selected from the group consisting of Ribonuclease III and Nuclease S1.

Both Ribonuclease III (“RNIII”) and Nuclease S1 (“S1”) can be purchased commercially. For example, RNIII is available from New England Biolabs, Inc. (www.neb.com) and S1 may be purchased from USB Corporation, 26111 Miles Road, Cleveland, Ohio 44128. S1 may also be purchased from Amersham, Invitrogen Corp., and Promega Corp.

By way of background, RNase III is a double-stranded RNA-specific endonuclease which will cleave dsRNA into fragments of 12-15 bp that have 2 base 3′ overhangs. It is typically used for mapping RNA structure. References: Robertson, H. D. et al. (1968) J. Biol. Chem. 243, 82; Lamontagne, B. et al. (2001) Curr. Issues Mol. Biol. Bol. 3, (pp71-78). San Diego: Academic Press.

S1 nuclease degrades single-stranded nucleic acids. It is typically used for mapping RNA transcripts and removing single-stranded tails. References: Berk, A. J. and Sharp, P. A. (1978) Proc. Natl. Acad. Sci. (USA) 75: 1274; Roberts, T. M. et al. (1979) Proc. Natl. Acad. Sci. (USA) 76: 760.

The fragments produced by the step of fragmenting the nucleic acid sample may, according to the present invention, have a variety of lengths. The range is very wide. Persons of skill in the art will recognize that the ideal fragment length will depend upon the particular application. Fragments can range up to 500 nucleotides. According to the present invention, fragments preferably have an average size of between about 20 to 500 nucleotides, more preferably between about 25 to 200 nucleotides and most preferably between about 50 to 100 nucleotides.

Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., .sup.3 H, .sup.125 I, .sup.35 S, .sup.14 C, or .sup.32 P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The label may be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an aviden-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are preferred and easily added during an in vitro transcription reaction. In a preferred embodiment, fluorescein labeled UTP and CTP are incorporated into the RNA produced in an in vitro transcription reaction as described above. cRNA, according to the present invention, is preferably labeled with biotin.

Also provided according to the present invention is a method for detecting hybridization of a nucleic acid sample to a nucleic acid array. This method has the following steps: providing a nucleic acid sample comprising mRNA transcripts of one or more genes; reverse transcribing the nucleic acid sample with a reverse transcriptase and a promoter consisting of oligo dT and a sequence encoding the phage T7 promoter to provide single stranded DNA template; synthesizing double stranded cDNA from the single stranded DNA template using DNA polymerase to provide cDNA template; transcribing the cDNA template with T7 RNA polymerase to provide cRNA; fragmenting the cRNA with an RNase to provide fragmented cRNA; and hybridizing said fragmented cRNA to a nucleic acid array. According to the present invention, the preceding method also preferably includes an additional step of end labeling the fragmented cRNA. Preferably, the end labeling is with biotin.

Also, according to the present invention, the step of transcribing the cDNA template may preferably be carried out in the presence of biotin labeled ribonucleotides to provide biotin labeled cRNA. Preferred embodiments with respect to the RNase enzyme and fragment size are as set forth above.

A nucleic acid array according to the present invention is any solid support having a plurality of different nucleotide sequences attached thereto or associated therewith. One preferred type of nucleic acid array that is useful in the present invention include those that are commercially available from Affymetrix (Santa Clara, Calif.) under the brand name GeneChip®. Example arrays are shown on the website at affymetrix.com.

In another aspect of the present invention, a method is provided for preparing cRNA for hybridization to an oligonucleotide probe array, having the steps of providing cRNA and fragmenting the cRNA with an RNase enzyme to provide fragmented cRNA. Preferably, the cRNA is labeled with biotin. Alternatively and preferably, the method can include an additional step of end-labeling the fragmented cRNA. Preferably, the end-labeling is with biotin. Preferred embodiments with respect to the RNase enzyme and fragment length are as set forth above.

In another aspect of the present invention, a method for labeling an RNA sample is presented, the method comprising providing an RNA sample; fragmenting said RNA sample with and RNAse enzyme to produce RNA fragments; and end-labeling said RNA fragments with a detectable label. Preferably, the RNA sample is selected from the group consisting of total RNA, mRNA and cRNA.

Preferably, the RNase enzyme is selected from the group consisting of Ribonuclease III, Nuclease S1, RNase A, RNase H, RNase T1, and Mung Bean nuclease. More preferably, the RNase enzyme Ribonuclease III.

RNA Fragments preferably have an average size of between about 20 to 500 nucleotides. More preferably, the fragments have an average size of between about 25 to 200 nucleotides. Still more preferably, the fragments have an average size of between about 50 to 100 nucleotides.

Preferably, the detectable label is biotin. It is also preferred that the step of end labeling is performed with the enzyme T4 RNA ligase. Methods, procedures and compounds for end labeling RNA fragments with biotin using T4 RNA ligase are presented U.S. application Ser. No. 10/617,992 filed Jul. 11, 2003, which is incorporated herein by reference for all purposes.

In another aspect of the present invention, the method described above for labeling an RNA fragment produced with an RNAse enzyme may be used, in accordance with the present invention, in a method to detect the presence of an RNA molecule in an RNA sample by hybridizing the labeled RNA fragments to a nucleic acid array. Preferably, the nucleic acid array is an oligonucleotide array. Other preferred embodiments of this aspect of the present invention are as described above.

### EXAMPLES

Fragmentation of cRNA Using Ribonuclease III or Nuclease S1

cRNA Fragmentation

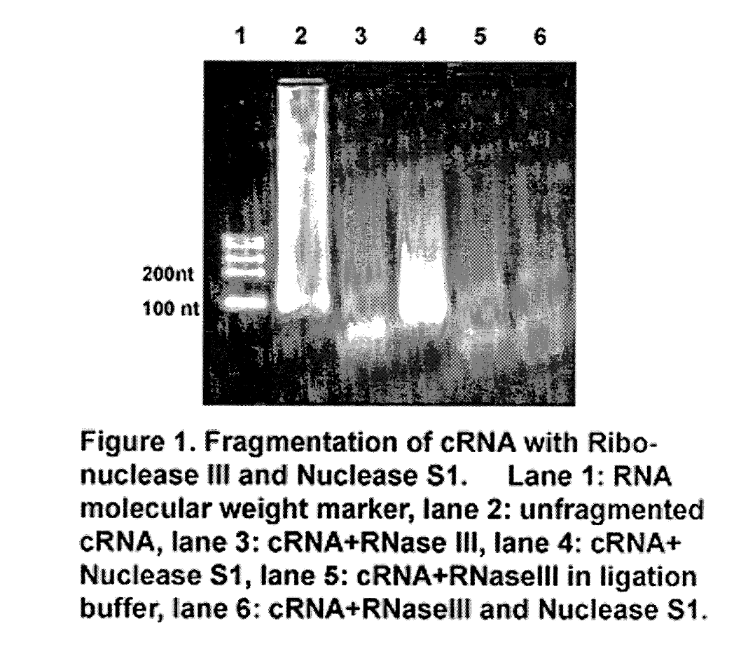

To test the efficacy of endonuclease digestion of cRNA, 1 μg of cRNA was incubated with 1 unit of RNase III or 1 U of Nuclease S1 or both in buffer containing 50 mM NaCl, 10 mM Tris, 10 mM MgCl₂ and 1 mM DTT, pH 7.9. RNase III digestion was also tried in buffer containing 50 mM TR is, 10 mM MgCl₂, 10 mM DTT, pH 8.0. Reactions were incubated at 37° C. for 30 minutes and resolved on a 2% agarose gel. As seen in FIG. 1, both RNase III (lanes 3, 5 and 6) and Nuclease S1 (lanes 4 and 6) efficiently fragment cRNA. RNase III digestion produces an average fragment size in the range of 50-200 nt, whereas Nuclease S1 produces an average fragment size between 100-200 nt.

End-Labeling Efficiency

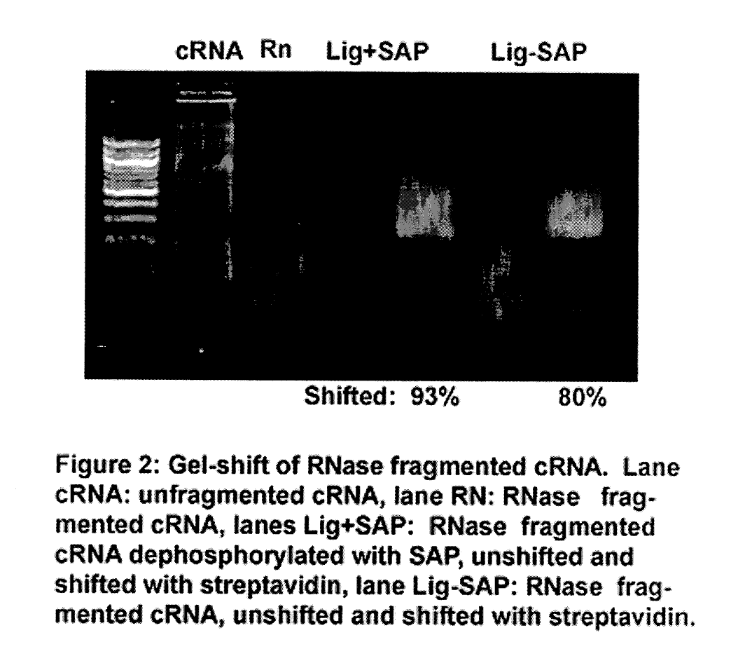

We compared the end-labeling efficiency of cRNA fragmented by RNase III and magnesium hydrolysis. For each sample 1 μg of cRNA was fragmented as described above. One sample was dephosphorylated with Shrimp alkaline phosphatase (SAP) after fragmentation. cRNA samples were end-labeled with biotin using T4 RNA ligase and pCp-biotin. Gel-shifted samples were incubated with streptavidin (1 mg/ml) prior to electrophoresis on a 10% acrylamide TBE gel. FIG. 2 shows the results of the labeling efficiency based on a gel shift assay. We found that 80-93% of dephosphorylated, RNase-fragmented cRNA was labeled with biotin while only 60-70% of the magnesium hydrolyzed RNA was labeled (data not shown).

Performance of RNase III Fragmented cRNA on DNA Microarrays

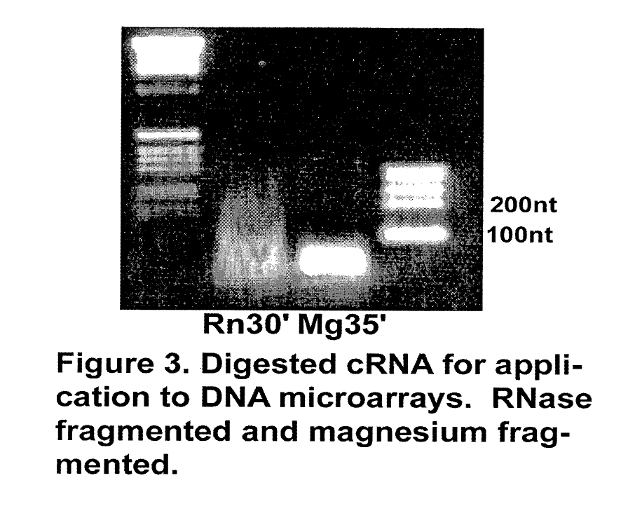

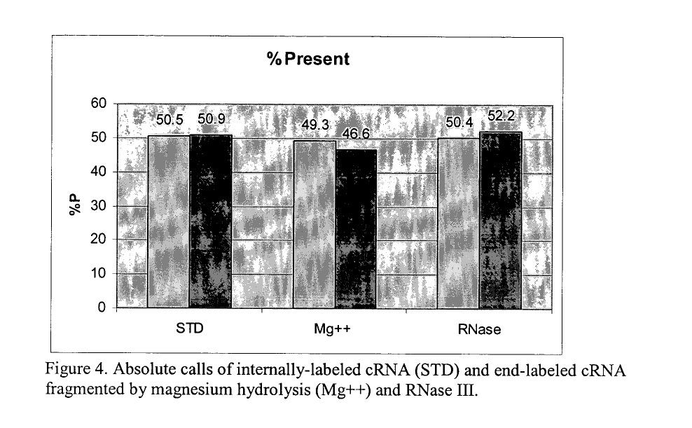

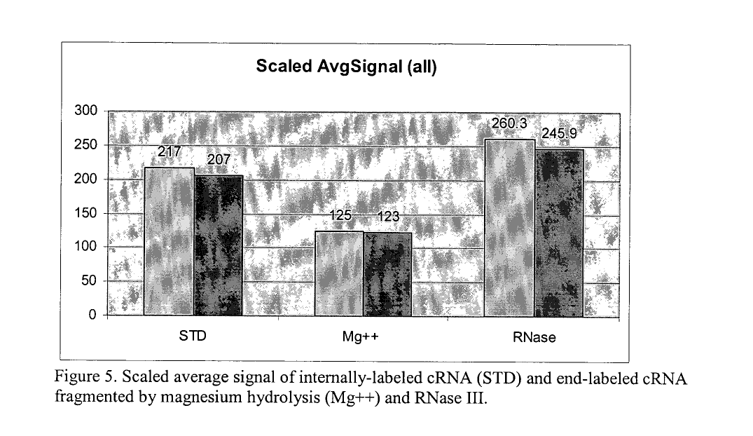

cRNA samples were fragmented as described above except that 11 μg of cRNA was fragmented, dephosphorylated, end-labeled and hybridized to GeneChip U133A arrays. FIG. 3 shows that the average size of RNase-fragmented cRNA is larger than magnesium hydrolyzed cRNA. The chip results shown in FIGS. 4 and 5 indicate that RNase-fragmented cRNA gives a higher absolute call rate (% P) and average signal compared to internally labeled cRNA or magnesium fragmented RNA.

The foregoing invention has been described in some detail by way of illustration and examples, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1. A method for preparing a nucleic acid sample for hybridization to a nucleic acid array, said method comprising:
providing a nucleic acid sample, said nucleic acid sample comprising mRNA;
amplifying the mRNA to produce cRNA; and
fragmenting the cRNA with an RNase enzyme to provide fragments.

2. The method of claim 1 further comprising the step of hybridizing said fragments to an oligonucleotide probe array.

3. The method of claim 1 wherein said RNase enzyme is selected from the group consisting of Ribonuclease III, Nuclease S1, RNase A, RNase H, RNase T1, and Mung Bean nuclease.

4. The method of claim 3 wherein said RNase enzyme is Ribonuclease III.

5. The method of claim 1 wherein said fragments have an average size of between about 20 to 500 nucleotides.

6. The method of claim 5 wherein said fragments have an average size of between about 25 to 200 nucleotides.

7. The method of claim 6 wherein said fragments have an average size of between about 50 to 100 nucleotides.

8. A method for detecting hybridization of a nucleic acid sample to a nucleic acid array, said method comprising:
providing a nucleic acid sample comprising mRNA transcripts of one or more genes;
reverse transcribing said nucleic acid sample with a reverse transcriptase and a promoter consisting of oligo dT and a sequence encoding the phage T7 promoter to provide single stranded DNA template;
synthesizing double stranded cDNA from said single stranded DNA template using DNA polymerase to provide cDNA template;
transcribing said cDNA template with T7 RNA polymerase to provide cRNA;
fragmenting said cRNA with an RNase to provide fragmented cRNA; and
hybridizing said fragmented cRNA to a nucleic acid array.

9. The method of claim 8 wherein said nucleic acid array is an oligonucleotide probe array attached to a support.

10. The method of claim 9 wherein said oligonucleotide probe array has at least about 100 probes per square centimeter.

11. The method of claim 8 wherein said RNase enzyme is selected from the group consisting of Ribonuclease III, Nuclease S1, RNase A, RNase H, RNase T1, and Mung Bean nuclease.

12. The method of claim 11 wherein said RNase enzyme is Ribonuclease III.

13. The method of claim 8 wherein said fragments have an average size of between about 20 to 500 nucleotides.

14. The method of claim 13 wherein said fragments have an average size of between about 25 to 200 nucleotides.

15. The method of claim 14 wherein said fragments have an average size of between about 50 to 100 nucleotides.

16. A method for preparing cRNA for hybridization to an oligonucleotide probe array, said method comprising:
providing cRNA;
fragmenting said cRNA with an RNase enzyme to provide fragmented cRNA.

17. The method of claim 16 wherein said RNase enzyme is selected from the group consisting of Ribonuclease III, Nuclease S1, RNase A, RNase H, RNase T1, and Mung Bean nuclease.

18. The method of claim 17 wherein said RNase enzyme is Ribonuclease III.

19. The method of claim 16 wherein said fragments have an average size of between about 20 to 500 nucleotides.

20. The method of claim 19 wherein said fragments have an average size of between about 25 to 200 nucleotides.

21. The method of claim 20 wherein said fragments have an average size of between about 50 to 100 nucleotides.

22. The method of claim 16 wherein said cRNA is labeled with biotin.

23. The method of claim 16 further comprising the step of end labeling said fragmented cRNA.

24. The method of claim 23 wherein said end labeling is with biotin.

25. A method for labeling an RNA sample comprising
providing an RNA sample;
fragmenting said RNA sample with an RNAse enzyme to produce RNA fragments; and
end-labeling said RNA fragments with a detectable label.

26. A method according to claim 25 wherein said RNA sample is selected from the group consisting of total RNA, mRNA and cRNA.

27. The method of claim 25 wherein said RNase enzyme is selected from the group consisting of Ribonuclease III, Nuclease S1, RNase A, RNase H, RNase T1, and Mung Bean nuclease.

28. The method of claim 27 wherein said RNase enzyme is Ribonuclease III.

29. The method of claim 25 wherein said fragments have an average size of between about 20 to 500 nucleotides.

30. The method of claim 29 wherein said fragments have an average size of between about 25 to 200 nucleotides.

31. The method of claim 30 wherein said fragments have an average size of between about 50 to 100 nucleotides.

32. The method of claim 25 wherein said detectable label is biotin.

33. The method according to claim 25 wherein said step of end labeling is performed with T4 RNA ligase.

34. A method for detecting the presence an RNA molecule in an RNA sample, said method comprising
providing an RNA sample;
fragmenting said cRNA with an RNase enzyme to provide RNA fragments;
end-labeling said RNA fragments with a detectable label to provide end-labeled RNA fragments; and
hybridizing said end-labeled RNA fragments to a nucleic acid array.

35. A method according to claim 34 wherein said nucleic acid array is an oligonucleotide array.

36. A method according to claim 34 wherein said RNA sample is selected from the group consisting of total RNA, mRNA and cRNA.

37. The method of claim 34 wherein said RNase enzyme is selected from the group consisting of Ribonuclease III, Nuclease S1, RNase A, RNase H, RNase T1, and Mung Bean nuclease.

37. The method of claim 36 wherein said RNase enzyme is Ribonuclease III.

38. The method of claim 34 wherein said fragments have an average size of between about 20 to 500 nucleotides.

39. The method of claim 38 wherein said fragments have an average size of between about 25 to 200 nucleotides.

40. The method of claim 39 wherein said fragments have an average size of between about 50 to 100 nucleotides.

41. The method of claim 34 wherein said detectable label is biotin.

42. The method according to claim 34 wherein said step of end labeling is performed with T4 RNA ligase.
